(19) United States
(12) Patent Application Publication
(10) Pub. No.: US 2009/0215717 A1
(43) Pub. Date: Aug. 27, 2009

(54) Sulfated oligosaccharides

(75) Inventors: Janos Kuszmann, Budapest (HU); Istvan Kurucz, Budapest (HU); Gabor Medgyes, Budapest (HU); Nicholas Bodor, Bal Harbour, FL (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Ivax Drug Research Institute Ltd., Budapest (HU)

(21) Appl. No.: 11/659,300

(22) PCT Filed: Aug. 5, 2005
(86) PCT No.: PCT/US2005/027921

§ 371 Date: Oct. 21, 2008

Related U.S. Application Data
(60) Provisional application No. 60/599,149, filed on Aug. 5, 2004.

(51) Int. Cl. A61K 31/7016; C07H 3/04; C07H 3/06; A61P 11/00; A61K 31/702

(52) U.S. Cl. 514/53; 536/123; 536/123.13; 514/61

(57) ABSTRACT

The invention relates to sulfated oligosaccharides, more particularly to new pharmaceutical uses of sulfated oligosaccharides. The invention provides a method of preventing, treating or alleviating the symptoms of acute and chronic’, inflammatory disorders of the airways of mammals—including asthma and asthma-related pathologies. The invention further provides use of a sulfated oligosaccharide in the preparation of a medicament for the treatment of acute and chronic inflammatory disorders of the airways of mammals. The invention yet further provides use of a sulfated oligosaccharide to preventing, treating or alleviating the symptoms of acute and chronic inflammatory disorders of the airways of mammals—including asthma and asthma-related pathologies.

### BACKGROUND OF THE INVENTION

1. Field of the Invention

The present invention relates to sulfated oligosaccharides, more particularly to new pharmaceutical uses of sulfated oligosaccharides.

The invention provides a method of preventing, treating or alleviating the symptoms of acute and chronic inflammatory disorders of the airways of mammals—including asthma and asthma-related pathologies.

The invention further provides use of a sulfated oligosaccharide in the preparation of a medicament for treatment of acute and chronic inflammatory disorders of airways of mammals.

The invention yet further provides use of a sulfated oligosaccharide to preventing, treating or alleviating the symptoms of acute and chronic inflammatory disorders of the airways of mammals—including asthma and asthma-related pathologies.

2. Summary of Related Art

Inflammation is a multi-step cascade process, any part of which may be the subject of potential therapeutic intervention. Briefly, inflammation entails the infiltration of immunologically competent cells (for example eosinophils, mast cells, activated T-lymphocytes) into the injury site where they, together with resident cells, release bioactive mediator substances (e.g., histamine, probes, a host of cytokines and chemokines), which increase the permeability of nearby blood vessel, attract and stimulate bystander cells. The altered permeability of vessels results in a fluid exudates forming at the injury site followed by a further influx of reactive leukocytes and their eventual efflux into the damaged area. (For an overview see, Trowbridge and Emling, Inflammation: A Review of the Process Quintessence Pub. Co., 1997). Secretion of collagen and mucus by, and proliferation of, resident cells (smooth muscle and epithelial cells or fibroblasts stimulated by the released mediators) establish the extension of pathological alterations (e.g., airway obstruction) and contribute to their development.

Inflammation is associated with a variety of pulmonary conditions including e.g., intrinsic or extrinsic asthma bronchiale, any inflammatory lung disease, acute or chronic bronchitis, pulmonary inflammatory reactions secondary to chronic bronchitis, chronic obstructive lung disease, pulmonary fibrosis, as well as any pulmonary condition in which white blood cells may play a role including, but not limited to, idiopathic pulmonary fibrosis and any other autoimmune lung disease. Asthma is perhaps one of the most common forms of pulmonary inflammation affecting the large and small airways of the lung. It impacts on 5% to 10% of the human population, resulting in an estimated 27 million patient visits, 6 million lost work-days, and 90.5 million days of restricted activity per year. The morbidity and mortality rates for asthma are growing worldwide (Plaut and Zimmerman, “Allergy and Mechanisms of Hypersensitivity” in Fundamental Immunology, 3rd Ed., Paul (ed.), Raven Press, New York, N.Y., at 1399 (1993)).

Conventional anti-asthma treatments have been predicated on the strict avoidance of all triggering allergens, which is inherently difficult to achieve, and on therapeutic regimens based on pharmacological agents having unfortunate side effects and suboptimal pharmacokinetic properties β2-adrenergic agonists used to treat bronchospasm have no effect on airway inflammation or bronchial hyperreactivity (Palmer et al., New Engl. J. Med. 331:1314 (1994)). also, regular or prolonged use of β2-adrenergic agonists is associated with poor control of asthma, increase in airway hyperresponsiveness to allergen, and reduced bronchoconstriction protection (Bhagat et al., Chest 108:1235 (1995)). Moreover, chronic use of β2-adrenergic agents alone, by causing down regulation of 2-adrenergic receptors, is suspected to worsen bronchial hyperreactivity. Theophylline (an anti-asthma methylxanthine) is characterized by substantial variability in its absorbance and clearance. Corticosteroids, while relatively safe in adult patients, are toxic for children, resulting in adrenal suppression and reduced bone density and growth (Woolock et al., am. Respir. Crit. Care Med. 153:1481 (1996)). Cromolyn, used to prevent asthmatic episodes, is effective in preventing an asthmatic reaction only if given prior to an attack (Volcheck et al., Postgrad Med. 104(3):127 (1998)). Antihistamines occasionally prevent or abort allergic asthmatic episodes, particularly in children, but often are only partially effective because histamines are only one of many inflammation associated mediators (Cuss, “The Pharmacology of Antiasthma Medications”, in Asthma as an Inflammatory Disease, O'Byrne, Ed., Dekker, Inc., New York, at 199 (1990)) and O'Byrne, “Airway Inflammation and Asthma”, in Asthma as an Inflammatory Disease, O'Byrne, Ed., Dekker, Inc., New York, N.Y., 143 (1990)).

Thus, current drug modalities suffer from a number of drawbacks. In general, conventional agents have a relatively short duration of action and may be partially or wholly ineffective when administered after antigen challenge occurs. Moreover, because of serious adverse effects associated with use of agents such as β2-adrenergic agonists and corticosteroids, therapeutic margins of safety with such agents are relatively narrow and patients using such agents must be carefully monitored (see e.g., WO 94/06783, WO 99/06025, U.S. Pat. Nos. 5,690,910 and 5,980,865). In a recent clinical study, with inhaled corticosteroids, only transient improvement occurred in the airways function of 5-11-year-old asthmatic children after the first year of therapy, with regression to that observed with placebo over the next 3 years (The Childhood Asthma Management Program Research Group, N. Engl. J. Med., 343:1054 (2000)). This observation can best be explained by remodeling changes (characteristic feature of asthma) occurring in the airways that are refractory to corticosteroids (Davies, Curr. Opin. Allergy Clin. Immunol., 1:67 (2001)).

The antiasthmatic activity of low molecular weight heparin as well as its sulfated degradation products containing uronic acid residues is well documented in the literature (U.S. Pat. No; 5,690,910; U.S. Pat. No. 5,980,865 and WO 02/083700).

Furthermore synthetic sulfated oligosaccharides were claimed to possess manyfold biological activities, such as anticoagulant and/or antithrombotic activity (see e.g. U.S. Pat. No. 6,271,215; WO 95/09637; WO 96/09828; U.S. Pat. No. 5,739,115, U.S. Pat. No. 5,721,357; U.S. Pat. No. 5,707,973; U.S. Pat. No. 4,496,550 and U.S. Pat. No. 6,271,215), and were used for the treatment of arteriosclerotic disorders (U.S. Pat. No. 5,447,919), inhibition of smooth muscle cell proliferation (U.S. Pat. No. 5,380,716), enhancement of wound healing (U.S. Pat. No. 4,912,093), lowering of blood triglyceride levels (U.S. Pat. No. 6,670,339), inhibition of cardiovascular disease (U.S. Pat. No. 6,638,916), for modulating metastasis activity (US 2003/0236223) and as complement inhibitors (JS 4,098,995).

### SUMMARY OF THE INVENTION

The present invention teaches that certain sulfated oligosaccharides, which are known in the literature and possess manyfold biological activities, are useful in treating inflammations of the airways. These sulfated oligosaccharides of the present invention have been found to reduce the bronchial airway hyper-responsiveness commonly associated with inflammations of the airways. Such oligosaccharides are also useful to suppress growth factor-induced proliferation of smooth muscle cells and inhibit allergen-induced mucus-secretion of airway epithelial cells thereby providing novel modalities for the treatment of airway obstruction.

### DETAIL DESCRIPTION OF THE INVENTION

The present invention discloses a method of preventing, treating or alleviating symptoms of acute and chronic inflammatory disorders of the airways of mammals using sulfated oligosaccharides. These oligosaccharides include compounds of a formula selected from the group consisting of

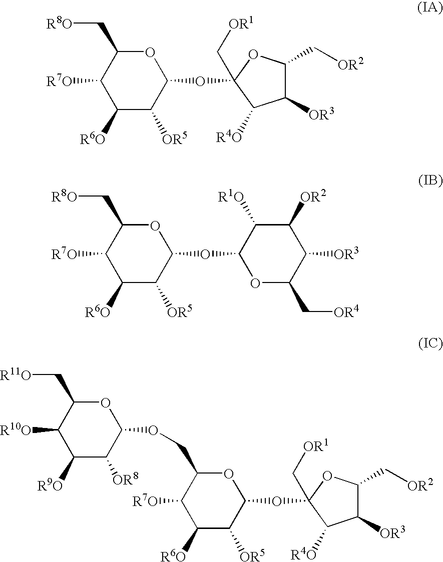

wherein R₁-R₁₁ groups are independently selected from the group consisting of C₁-C₄ alkyl, —H, —SO₃M wherein M is a pharmaceutically acceptable cation, aryl, C₆-C₁₂ arylalkyl, wherein at least one of R₁-R₁₁ represents —SO₃M, or pharmaceutically acceptable salts thereof. Simple and complex mixtures of compounds formulas corresponding to (IA), (IB) and (IC) are also acceptable.

This preferred embodiment of this method comprises administration to a patient of an effective amount of at least one sulfated oligosaccharide of formula (II), more specifically (IIA), (IIB) or (IIC),

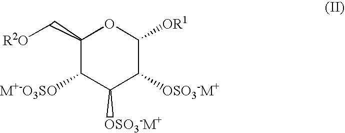

wherein R¹ represents a pharmaceutically acceptable salt of a fully sulfated β-D-fructofuranoside unit and R² represents a pharmaceutically acceptable salt of a —SO₃H group (IIA),

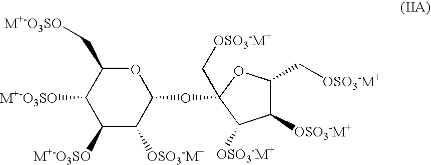

or R¹ represents a pharmaceutically acceptable salt of a fully sulfated α-D-glucopyranoside unit and R² represents a pharmaceutically acceptable salt of sulfite group (IIB),

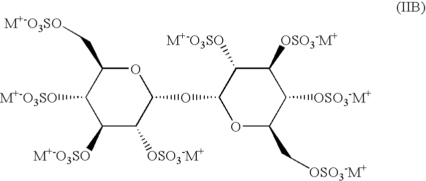

or a pharmaceutically acceptable salt of a fully sulfated α-D-galactopyranosyl unit (IIC),

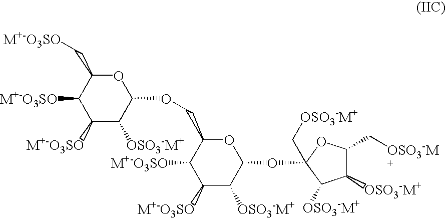

and M⁺ represents a pharmaceutically acceptable cation.

Preferred embodiments of the compounds of formula (II) of the present invention are alkali metal-, alkaline-earth metal- and ammonium salts of sucrose octasulfate, trehalose octasulfate and raffinose undecasulfate respectively e.g. sucrose octasulfate octa sodium salt, trehalose octasulfate octa potassium salt and raffinose undecasulfate undeca potassium salt.

Compounds (IIA), (IIB) and (IIC) and the preparation thereof are known from the literature [see e.g. (IA): U.S. Pat. No. 5,767,104; U.S. Pat. No. 6,271,215; WO 90/12561; HU 195 832 and K. Ochi et al., Chem. Pharm. Bull 28 (1980) 638-641, (IB): U.S. Pat. No. 5,906,924, (IC): U.S. Pat. No. 4,098,995].

“Pharmaceutically acceptable cation” refers to cations that may be used to form non-toxic salts of the compounds of formula (I). Such cations are well known in the art and can be found described in Remington: The Science and Practice of Pharmacy, Al Gennaro, ed., 20th Ed., Lippincott Williams & Wilkins, 2000. Nonlimiting examples of pharmaceutically acceptable cations include alkali metal (e.g. sodium, potassium, lithium) ions, alkaline-earth metal (e.g. calcium, magnesium) ions and other pharmaceutically acceptable metal ions (e.g. zinc and aluminium). Other examples of such cations include, without limitation, ammonium cations derived from ammonia or a pharmaceutically acceptable primary, secondary or tertiary amine.)

Especially preferred pharmaceutically acceptable cations are the alkali metal, in particular sodium or potassium ions.

As used in this specification, the singular forms “a”, “an” and “the” specifically also encompass the plural forms of the terms to which they refer, unless the content clearly dictates otherwise. For example, reference to “a modulator” includes mixtures of modulators.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, unless specifically indicated otherwise, the word “or” is used in the “inclusive” sense of “and/or” and not the “exclusive” sense of “either/or.”

As used herein, the terms “treating” or “treatment” are used to indicate reducing, alleviating, preventing, inhibiting the development of and/or reversing the symptoms of a condition. Conditions to be treated by the methods and compositions of the invention include any condition characterized by, or including, acute and chronic inflammatory disorders of the airways. Hence, the terms “inflammatory disorder” or “inflammatory disorders of the airways” encompass any inflammatory lung disease, including asthma, intrinsic or extrinsic asthma bronchiale, acute chronic bronchitis, allergic rhinitis, pulmonary inflammatory and structural reactions secondary to chronic bronchitis, chronic obstructive lung disease, pulmonary fibrosis. The invention is also useful for any pulmonary condition in which white blood cells and airway remodeling may play a role including but not limited to idiopathic pulmonary fibrosis and any other autoimmune lung disease.

By “asthma” is meant a condition of allergic origins, the symptoms of which include continuous or paroxysmal labored breathing accompanied by wheezing, a sense of constriction in the chest, and often attacks of coughing or gasping. By “asthma-related pathology” is meant a condition whose symptoms are predominantly inflammatory in nature with associated bronchospasm. Hence, both asthma and asthma-related pathologies are characterized by symptoms that include narrowing of airways, due in varying degrees to contraction (spasm) of smooth muscle, edema of the mucosa, including that of the upper airways and mucus in the lumen of the bronchi and bronchioles. Non-limiting representative examples of “asthma-related pathologies” include non-asthmatic conditions characterized by airway hyperresponsiveness (e.g., chronic bronchitis, emphysema, cystic fibrosis and respiratory distress).

Compositions and methods taught herein are exemplified, for asthma. However, the invention should not be construed as limited to this particular pulmonary disease. Asthma offers the advantage of having been studied extensively and provides several accepted models to evaluate the invention. It is known that sensitization and allergen challenge leads to airway hyperresponsiveness to various agonists. Hence, acetylcholine, known as a spasmogenic agent, is capable of inducing larger contractions of the muscle cells in tissues obtained from the trachea of sacrificed animals (which had been sensitized to provoke airway hyper-responsiveness) than from control animals following allergen challenge (see, e.g. Tokuoka et al., Br. J. Pharmacol. 134:1580 (2001); Nakata et al., Int. Immunol. 13:329 (2001); Emala and Hirshman, Monogr. Allergy 33:35 (1996)).

The most prominent characteristic of asthma is bronchospasm, or narrowing of the airways. Asthmatic patients have prominent contraction of the smooth muscles of large and small airways, increased mucus production, and increased inflammation (Plaut and Zimmerman, supra). The inflammatory response in asthma is typical for tissues covered by a mucosa and is characterized by vasodilation, plasma exudation, recruitment of inflammatory cells such as neutrophils, monocytes, macrophages, lymphocytes, and eosinophils to the sites of inflammation, and the release of inflammatory mediators by resident tissue cells (e.g., mast cells or airways epithelial cells) or by migrating inflammatory cells (Hogg, “Pathology of Asthma”, in Asthma as an Inflammatory Disease, O'Byrne (ed.), Marcel Dekker, Inc., New York, N.Y., at 1 (1990)). Asthma may be triggered by a variety of causes such as allergic reactions, a secondary response to infections, industrial or occupational exposures, ingestion of certain chemicals or drugs, exercise (Hargreave et al., J. Allergy Clin. Immunol. 83:1013 (1986)).

The compounds of formula (II), more specifically (IIA), (IIB) and (IIC) according to the invention have also been found effective to decrease mucus production of bronchial epithelial cells and to inhibit growth factor mediated proliferation of smooth muscle cells.

An increase in bronchial hyperreactivity (AHR), the hallmark of a more severe form of asthma, can be induced by both airway antigenic and non-antigenic stimuli. Late phase response and persistent hyperresponsiveness in allergen-induced asthma have been associated with the recruitment of leukocytes, and particularly eosinophils, to inflamed lung tissue (Abraham et al., Am. Rev. Respir. Dis. 138:1565 (1988)). Eosinophils release several inflammatory mediators including 15-HETE, leukotriene C4, PAF, cationic proteins, eosinophil peroxidase.

The terms “antigen” and “allergen” are used interchangeably to describe those molecules, such as dust or pollen that can induce an allergic reaction and/or induce asthmatic symptoms in an individual suffering from asthma. Thus, an asthmatic individual “challenged” with an allergen or an antigen is exposed to a sufficient amount of the allergen or antigen to induce an asthmatic response. The compounds of formula (II), more specifically (IIA), (IIB) and (IIC) according to the invention have been found effective to treat AHR subsequent to ovalbumin sensitization and antigen challenge.

The biological activity of the compounds of formula (II) in different animal models is demonstrated below on the example of sucrose octasulfate octa sodium salt (IIA):

### Model 1.

Examination of the effect of locally administered sulfated oligosaccharides on airways' hyper-responsiveness ex vivo.

Inflammation of the airways may lead to bronchial hyper-responsiveness, which is a characteristic feature of asthma.

Brown Norway (BN) rats were actively sensitized to ovalbumin (OA) by a subcutaneous injection of 0.5 ml of OA/Al(OH)₃ gel mixture (2 mg OA+10 g Al(OH)₃/100 ml saline) on day 1 with subsequent subcutaneous injections (10 mg OA+10 g Al(OH)₃/100 ml saline) given on days 14 and 21. On day 28, animals received the compound of formula (IIA) intratracheally (0.01 or 1.0 mg/kg dose) 2 hours before antigen challenge. Antigen challenge was performed by inhalation of nebulised ovalbumin (1% antigen solution administered in a TSE inhalation system for 1 hour). Animals were sacrificed 48 hours post antigen challenge wherein the tracheas were removed to an organ bath. Dissected tracheas were allowed to equilibrate for 30 minutes before measuring tracheal spasmogenic response curves to acetylcholine (Ach).

As shown in Table 1 ovalbumin challenge of sensitized animals in this model caused a significant tracheal hyper-reactivity to acetylcholine, when the response to the spasmogenic agent was determined 48 h after antigen challenge. The compound described in formula (IIA) in both of the applied doses notably decreased this elevation.

TABLE 1
Effect of antigen challenge and intratracheal
pretreatment with compound of formula (IIA) on
the tracheal contraction to acetylcholine in BN-rats

| | | | Examined compound | |
|---|---|---|---|---|
| Parameters | Control | Placebo | 0.01 mg/kg | 1.0 mg/kg |
| ED₅₀* | 5.16 ± 0.08 | 6.10 ± 0.30 | 4.73 ± 0.50 | 4.98 ± 0.30 |
| p | <0.001 | | 0.022 | 0.011 |
| MAX** | 100 ± 0 | 220 ± 30 | 167 ± 36 | 131 ± 22 |
| p | <0.001 | | NS*** | 0.007 |

*log M acetylcholine (Ach), causing 50% contraction relative to control (mean ± SEM)
**Contraction at maximal Ach concentration relative to control (mean ± SEM)
***Non-significant

### Model 2.

### Examination of the Effect of Sulfated Oligosaccharides on the Allergen Stimulated Mucus Production of Airways Epithelial Cells.

In a sensitized animal antigenic challenge results in mucus production of airways epithelial cells, which is a characteristic feature of allergic asthma.

Sensitized BN rats were treated intratracheally with 0.01 or 1.0 mg/kg dose of compound of formula (IIA), two hours before antigenic challenge, using a similar protocol described in Model 1. Lungs were collected 48 hours after challenge and were fixed in 8% phophospate buffered formaldehyde. Samples were then processed for histochemistry routinely. 5 μm thick sections were stained with periodic-acid-Schiff (PAS) reagents and were counterstained with haematoxylin-eosine. On the sections each epithelial cells of the airways were counted in the whole preparation at a magnification of 400×. The number of PAS(+) [mucus producing] epithelial cells was expressed as the ratio of the to a number of epithelial cells.

As it is shown in Table 2, allergen challenge stimulates the mucus production of airways epithelial cells (control vs. challenge). At the dose of 0.01 mg/kg the compound notably, at the higher dose statistically significantly decreased the number of PAS(+), mucus producing cells.

TABLE 2
Effect of antigen challenge and intratracheal treatment
with compound of formula (IIA), on the allergen induced
mucus production of airways epithelial cells in BN rats

| Groups | Dose mg/kg | %* | p-value |
|---|---|---|---|
| Control | | 2.9 ± 0.9 | <0.001 |
| Challenge | | 27.6 ± 3.5 | — |
| Treated | 0.01 mg/kg | 19.2 ± 2.7 | NS |
| | 1.0 mg/kg | 7.3 ± 1.4 | <0.001 |

*number of PAS(+) cells as percent of total number of cells (average ± SEM)

### Model 3.

### Examination of the Effect of Sulfated Oligosaccharides on the Extent of Perivascular Oedema Developed in Asthmatic Lung Tissue.

In a sensitized animal antigen challenge, as a result of the developing inflammatory processes, increases the permeability of the blood vessels resulting in plasma excudation around the periphery of the vasculature.

Sensitized BN rats were treated intratracheally with 0.01 and 1.0 mg/kg dose of compound of formula (IIA), two hours before antigenic challenge, using a similar protocol described in Model 1. Lungs were collected 48 hours after challenge and were fixed in 8% phosphate buffered formaldehyde. Samples were then processed for histochemistry routinely. 5 pin thick sections were stained with periodic-acid-Schiff (PAS) reagents and were counterstained with haematoxylin-eosine. On the sections the areas of the connective tissue around the vasculare were determined and expressed as a ratio of the area of the corresponding blood vessel itself.

As it is shown in Table 3, allergen challenge causes aedema around the vasculature, the extent of which was significantly decreased by the higher dose of the examined compound.

TABLE 3
Effect of antigenic challenge and intratracheal
treatment with compound of formula (IIA), on
the extent of developing oedema in BN rats

| Groups | Dose mg/kg | Oedema* | p-value |
|---|---|---|---|
| Control | | 71 ± 7 | <0.001 |
| Challenge | | 224 ± 17 | — |
| Treated | 0.01 mg/kg | 177 ± 10 | NS |
| | 1.0 mg/kg | 119 ± 7 | <0.001 |

*area of oedema relative to area of vasculature (average ± SEM)

### Model 4.

### Examination of the Effect of Sulfated Oligosaccharides on the Extent of Eosinophilia Developed in the Asthmatic Lung.

In a sensitized animal antigenic challenge results in the infiltration of eosinophils into the lung and this phenomenon is one of the most typical feature of asthma.

Sensitized BN rats were treated intratracheally with 0.01 and 1.0 mg/kg dose of compound of formula (IIA); two hours before antigenic challenge, using a similar protocol described in Model 1. Bronchoalveolar lavage fluids (BALF) were collected 48 hours after challenge and after staining, the number of eosinophils were determined.

As it is shown in Table 4, allergen challenge causes an increase in the number of eosinophils in the bronchioalveolar lavage. Treatment with compound of formula (IIA), already at the smallest dose decreases the extent of it, at higher doses the decrease become statistically significant.

TABLE 4
Effect of antigen challenge and intratracheal treatment
with compound of formula(IA), on the extent of the
developing eosinophilia in the BALF of BN rats

| Groups | Dose mg/kg | Eosinophil* | p-value |
|---|---|---|---|
| Control | | 0.35 ± 0.04 | <0.01 |
| Challenge | | 1.00 ± 0.16 | — |
| Treated | 0.01 mg/kg | 0.89 ± 0.13 | NS |
| | 1.0 mg/kg | 0.70 ± 0.08 | <0.05 |

*eosinophil number/ml BALF (average ± SEM)

### Model 5.

### IP-3 Receptor Antagonistic Effect of Sulfated Oligosaccharides

The sulfated oligosaccharides of the present invention, depending on their chemical structure, inhibit the binding of inositol-1,4,5-trisphophate (1-3) to its receptor in microsomal membrane preparations. As IP-3 is a messenger molecule playing distinguished role in the activation of different cells, interfering with this function can explain the anti-asthmatic effect of these sulfated oligosaccharides.

The IP-3 antagonist effect of the sulfated oligosaccharides was determined using rat cerebellum membrane preparations according to Worley et al. (JBC 262, 12132, 1987). As is seen in Table 5, all the compounds described in formula (IIA), (IIB) and (IIC) possess significant IP-3 antagonist activity.

TABLE 5
IP-3 receptor antagonistic effect of sulfated oligosaccharides

| | IC₅₀ (μg/ml) | Average IC₅₀ |
|---|---|---|
| Compound | Average ± SEM (n) | (nM) |
| IIA | 0.26 ± 0.05 (4) | 208 |
| IIB | 0.24 ± 0.02 (3) | 194 |
| IC | 0.43 ± 0.05 (3) | 238 |

Notes:
IIA = sucrose octasulfate octa sodium salt
IIB = trehalose octasulfate octa potassium salt
IIC = raffinose undecasulfate undeca potassium salt

The compounds according to the invention are optimally formulated in a pharmaceutically acceptable vehicle with any of the well-known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention.

The compositions of the invention can be administered, by standard routes (e.g. oral, inhalation, rectal, nasal, topical, including buccal and sublingual, or parenteral, including subcutaneous, intramuscular, intravenous, intradermal, transdermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powder and aerosols. The present invention encompasses liquid and powdered compositions suitable for nebulization and intrabronchial use, or aerosol compositions administered via an aerosol unit dispensing metered doses (“MDI”). Particularly preferred devices contemplated are described in U.S. Pat. No. 5,447,150.

The active ingredient may be formulated in an aqueous pharmaceutically acceptable inhalant vehicle, such as, for example, isotonic saline or bacterostatic water and other types of vehicles that are well known in the art The solutions are administered by means of a pump or squeeze-actuated nebulized spray dispenser, or by any other conventional means for causing or enabling the requisite dosage amount of the liquid composition to be inhaled into the patient's lungs.

Powder compositions containing the anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule, which may be inserted by the patient into a device that punctures the capsule and blows the powder out in a steady steam.

Aerosol formulations for use in the subject method typically include propellants, surfactants, and co-solvents and may be filled into conventional aerosol containers that are closed by a suitable metering valve.

For oral administration, the anti-inflammatory compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

Formulations of compositions of the present invention suitable for nasal administration, wherein the carrier is a solid, include a coarse powder having a particle size, for example, in the range of 20 to 500 microns which is administered in the manner in which snuff is administered, i.e. by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention. Semi-liquid formulations, such as a nasal gel, are also suitable.

Formulations of compositions suitable for parenteral administration include aqueous and non-aqueous sterile injection solutions which may contain antioxidants, stabilizers, buffers, bacteriostats and solutes which render the formulation isotonic with the blood of the intended recipient, and aqueous and non-aqueous sterile suspensions which may, include suspending agents and thickening agents.

The pharmaceutical compositions of the present invention are intended for use with any mammal that may experience the benefits of the methods of the invention. Foremost among such mammals are humans, although the invention is not intended to be so limited, and is applicable to veterinary uses. Thus, in accordance with the invention, “mammal” or “mammal in need” include humans as well as non-human mammals, particularly domesticated animals including, without limitation, cats, dogs and horses.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another anti-asthmatic compound (e.g., corticosteroid). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect. Clinical changes relevant to assess the therapeutic effect of treatment according to the invention include reduction in the characteristic symptoms and signs of asthma and related pathologies (e.g., dyspnea, wheezing, cough, bronchial hypersensitivity airway remodeling) and improvement of pulmonary function tests. These are based upon patients symptoms and physician's observations.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

For local administration by inhalation for example, contemplated therapeutically effective amounts are from about 0.1 μg/day to about 1000 μg/kg/day when administered systemically (e.g., orally administered). In an embodiment of the invention, when systemically administered, therapeutically effective amounts are from about 0.5 μg/kg/day to about 200 μg/kg/day.

Dosage forms and frequency of administration of the same will depend on conventional factors routinely considered by one of skill in the field to obtain therapeutically effective amounts as discussed above in a given mammal. Hence, a practitioner will consider the condition being treated, the particular compound of the invention being administered, route of administration, and other clinical factors such as age, weight and condition of the mammal as well as convenience and patient compliance.

It will be appreciated by those of skill in the art that the number of administrations of the compounds according to the invention will vary from patient to patient based on the particular medical status of that patient at any given time.

When applicable (such as for the treatment of asthma, for example) the compound according to this aspect of the invention, may be administered prior to, at the same time, or after the mammal has been exposed to an antigen. In addition, the timing of the administration of the compound of the invention with relation to the exposure to an antigen will vary from mammal to mammal depending on the particular situation. A skilled practitioner will optimize administration by careful monitoring the patient while altering the timing and/or the order of administration of the compound of the invention. Hence, it will be understood that the mammal need not suffer from a pulmonary inflammation to benefit from the invention. The compounds of the invention may be administered prophylactically to individuals predisposed to develop asthma and/or an asthma-related pathology. For example, an individual allergic to pollen may be administered a compound of the invention (e.g., by oral administration) on a daily basis and/or prior to going to a pollen-rich area (e.g., a garden). Likewise, an individual with only a family history of asthmatic attacks may be administered the compounds of the invention prophylactically—to prevent or inhibit possible onset of such an asthmatic attack.

### EQUIVALENTS

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

1. A method for treating symptoms of acute or chronic inflammatory disorders of mammalian airways comprising the step of administering to a mammal a therapeutically effective amount of a compound of a formula selected from the group consisting of compounds of formulas (IA), (IB) and (IC), 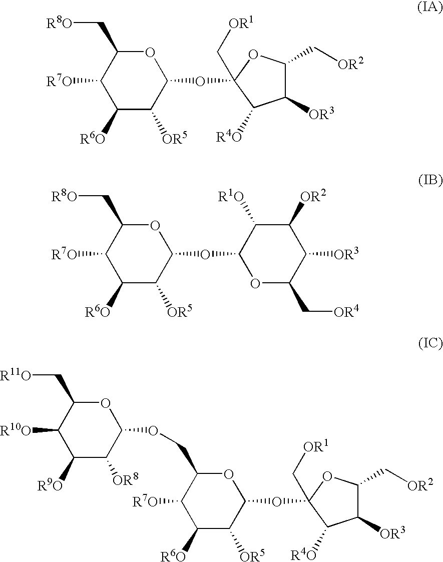
wherein R₁-R₁₁ groups are independently selected from the group consisting of C₁-C₄ alkyl, —H, —SO₃M wherein M is a pharmaceutically acceptable cation, amyl, C₆-C₁₂ arylalkyl, wherein at least 50% of R₁-R₁₁ represents —SO₃M, or pharmaceutically acceptable salts thereof.

2. The method of claim 1 wherein all R¹-R¹¹ represent —SO₃M wherein M is a pharmaceutically acceptable cation.

3. The method of claim 2 wherein said compound is selected from the group consisting of sucrose octasulfate octasodium salt, trehalose octasulfate octapotassium salt and raffinose undecasulfate undecapotassium salt.

4. A method for treating symptoms of acute or chronic inflammatory disorders of mammalian airways comprising the step of administering to mammal a therapeutically effective amount of a compound of a formula selected from the group consisting of compounds of formulas (IIA), (IIB) and (IIC), 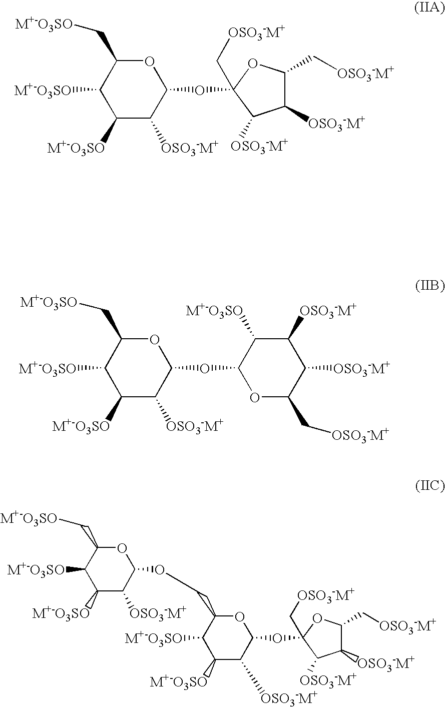
wherein M represents a pharmaceutically acceptable cation.

5. The method of claims 1 or 4 wherein said inflamatory disorder is an allergic inflamatory disorder.

6. The method according to claim 5, wherein the allergic inflammatory disorder of the airways is selected from the group consisting of asthma, allergic rhinitis, intrinsic or extrinsic asthma bronciale, acut or chronic bronchitis, chronic obstructive lung disease, and pulmonary fibrosis.

7. The method of claim 5 wherein said allergic inflamatory disorder is selected from the group consisting of idiopathic pulmonary fibrosis and autoimmune lung disease.

8. The method of claim 1 or 4 wherein said compound is administered as a single or multiple dose.

9. The method of claims 1 or 4 wherein said compound is administered together with a pharmaceutically acceptable carrier or diluent in the form of a composition.

10. A compound of formula selected from the group consisting of (IA), (IB) and (IC), 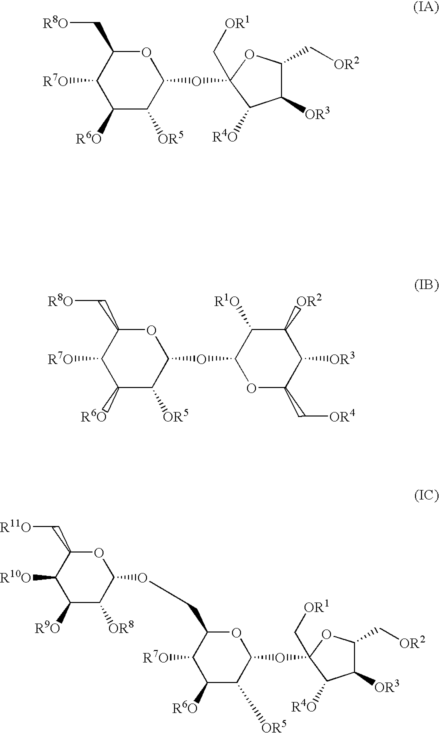
wherein at least one of R₁-R₁₁, groups is selected from the group consisting of C₁-C₄ alkyl, —H, aryl, C₆-C₁₂ arylalkyl, —SO₃M wherein M is a pharmaceutically acceptable cation, wherein not all of R¹-R¹¹ is —SO₃M.

11. A mixture of compounds of formulas selected from the group consisting of (IA), (IB) and (IC),
wherein at least one of R₁-R₁₁ groups is selected from the group consisting of C₁-C₄ alkyl, —H, aryl, C₆-C₁₂ arylalkyl, —SO₃M wherein M is a pharmaceutically acceptable cation, wherein at least 50% of R₁-R₁₁ represents —SO₃M wherein M is a pharmaceutically acceptable cation.

12. Use of a compound of formula (IA), (IB), (IC), 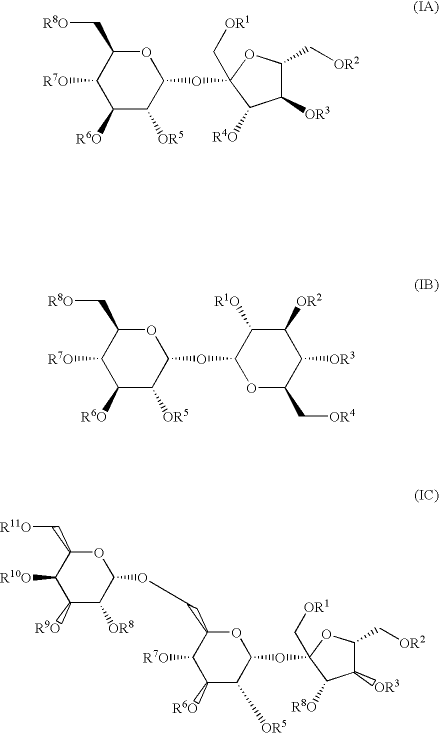
wherein R₁-R₁₁, groups are independently selected from the group consisting of C₁-C₄ alkyl, —H, —SO₃M wherein M is a pharmaceutically acceptable cation, aryl, C₆-C₁₂ arylalkyl, wherein at least 50% of R₁-R₁₁ represents —SO₃M, or pharmaceutically acceptable salts thereof, for treating the symptoms of acute and chronic inflammation disorders of the airways of mammals.

13. Use of a compound of formula (IA), (IB), (IC), 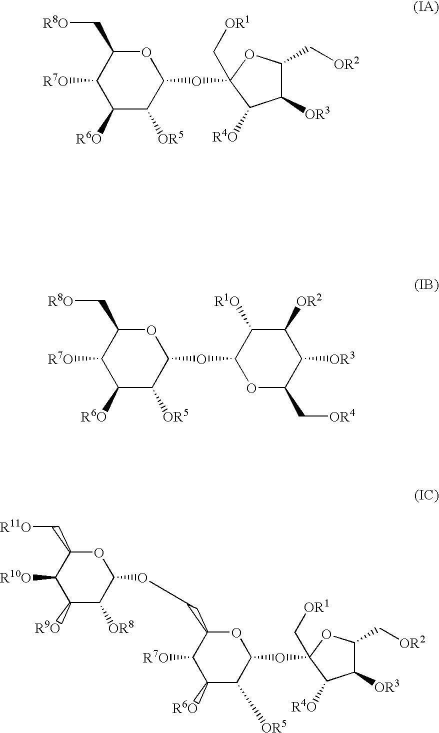
wherein R₁-R₁₁ groups are independently selected from the group consisting of C₁-C₄ alkyl, —H, —SO₃M wherein M is a pharmaceutically acceptable cation, aryl, C₆-C₁₂ arylalkyl, wherein at least 50% of R₁-R₁₁, represents —SO₃M, or pharmaceutically acceptable salts thereof, for preparation of a medicament suitable for treating the symptoms of acute and chronic inflammatory disorders of airways of mammals.

14. Use of a compound formula (IIA), (IIB) or (IIC) 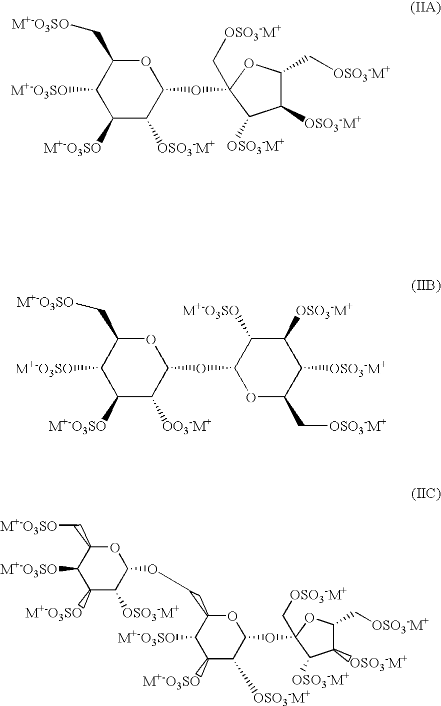
wherein M represents a pharmaceutically acceptable cation, for treating the symptoms of acute and chronic inflammatory disorders of the airways of mammals.

15. Use of a compound formula (IIA), (IIB) or (IIC) 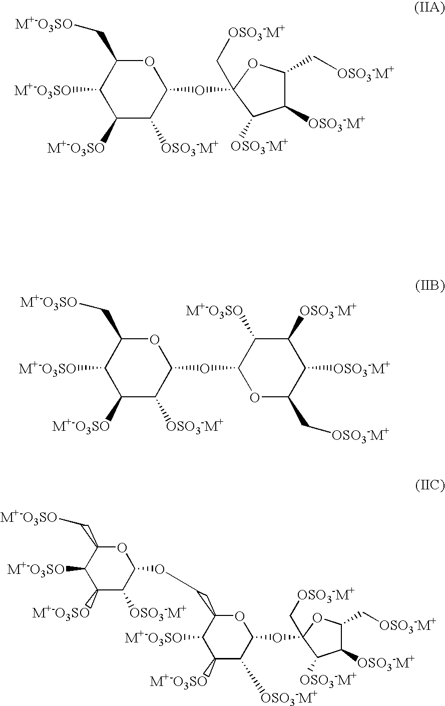
wherein M represents a pharmaceutically acceptable cat for preparation of a medicament suitable for treating the symptoms of acute and chronic inflammatory disorders of airways of mammals.

16. The use according to claim 13 or 15 for the preparation of a medicament suitable for treating allergic inflammatory disorders of airways.

17. The use of claim 16, wherein the allergic inflammatory disorder of airways is selected from the group consisting of asthma, allergic rhinitis, intrinsic or extrinsic asthma bronciale, acute or chronic bronchitis, chronic obstructive lung disease, and pulmonary fibrosis.

18. The use of claim 16, wherein the allergic inflammatory disorder of airways is selected from the group consisting of idiopathic pulmonary fibrosis and autoimmune lung disease.
